(19) United States
(12) Patent Application Publication
(10) Pub. No.: US 2004/0157828 A1
(43) Pub. Date: Aug. 12, 2004

(54) Protease inhibitors

(76) Inventors: Ren Xie, Collegeville, PA (US); Dennis S Yamashita, Collegeville, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/478,619

(22) PCT Filed: May 15, 2002
(86) PCT No.: PCT/US02/15376

(51) Int. Cl. C 07D 4 5/14; A61K 31/55

(52) U.S. Cl. 514/217.04; 540/597

(57) ABSTRACT

The present invention provides 4-amino-azepan-3-one protease inhibitors and pharmaceutically acceptable salts, hydrates and solvates thereof which inhibit proteases, including cathepsin K, pharmaceutical compositions of such compounds, novel intermediates of such compounds, and methods for treating diseases of excessive bone loss or cartilage or matrix degradation, including osteoporosis; gingival disease including gingivitis and periodontitis; arthritis, more specifically, osteoarthritis and rheumatoid arthritis; Paget's disease; hypercalcemia of malignancy; and metabolic bone disease, comprising inhibiting said bone loss or excessive cartilage or matrix degradation by administering to a patient in need thereof a compound of the present invention.

### FIELD OF THE INVENTION

This invention relates in general to 4-amino-azepan-3-one protease inhibitors, particularly such inhibitors of cysteine and serine proteases, more particularly compounds which inhibit cysteine proteases, even more particularly compounds which inhibit cysteine proteases of the papain superfamily, yet more particularly compounds which inhibit cysteine proteases of the cathepsin family, most particularly compounds which inhibit cathepsin K. Such compounds are particularly useful for treating diseases in which cysteine proteases are implicated, especially diseases of excessive bone or cartilage loss, e.g., osteoporosis, periodontitis, and arthritis.

### BACKGROUND OF THE INVENTION

Cathepsins are a family of enzymes which are part of the papain superfamily of cysteine proteases. Cathepsins B, H, L, N and S have been described in the literature. Recently, cathepsin K polypeptide and the cDNA encoding such polypeptide were disclosed in U.S. Pat. No. 5,501,969 (called cathepsin O therein). Cathepsin K has been recently expressed, purified, and characterized. Bossard, M. J., et al., (1996) J. Biol. Chem. 271, 12517-12524; Drake, F. H., et al., (1996) J. Biol. Chem. 271, 12511-12516; Bromme, D., et al., (1996) J. Biol. Chem. 271, 2126-2132.

Cathepsin K has been variously denoted as cathepsin O or cathepsin O2 in the literature. The designation cathepsin K is considered to be the more appropriate one.

Cathepsins function in the normal physiological process of protein degradation in animals, including humans, e.g., in the degradation of connective tissue. However, elevated levels of these enzymes in the body can result in pathological conditions leading to disease. Thus, cathepsins have been implicated as causative agents in various disease states, including but not limited to, infections by pneumocystis carinii, trypsanoma cruzi, trypsanoma brucei brucei, and Crithidia fusiculata; as well as in schistosomiasis, malaria, tumor metastasis, metachromatic leukodystrophy, muscular dystrophy, amytrophy, and the like. See International Publication Number WO 94/04172, published on Mar. 3, 1994, and references cited therein. See also European Patent Application EP 0 603 873 A1, and references cited therein. Two bacterial cysteine proteases from P. gingivallis, called gingipains, have been implicated in the pathogenesis of gingivitis. Potempa, J., et al. (1994) Perspectives in Drug Discovery and Design, 2, 445-458.

Cathepsin K is believed to play a causative role in diseases of excessive bone or cartilage loss. Bone is composed of a protein matrix in which spindle- or plate-shaped crystals of hydroxyapatite are incorporated. Type I collagen represents the major structural protein of bone comprising approximately 90% of the protein matrix. The remaining 10% of matrix is composed of a number of non-collagenous proteins, including osteocalcin, proteoglycans, osteopontin, osteonectin, thrombospondin, fibronectin, and bone sialoprotein. Skeletal bone undergoes remodelling at discrete foci throughout life. These foci, or remodelling units, undergo a cycle consisting of a bone resorption phase followed by a phase of bone replacement.

Bone resorption is carried out by osteoclasts, which are multinuclear cells of hematopoietic lineage. The osteoclasts adhere to the bone surface and form a tight sealing zone, followed by extensive membrane ruffling on their apical (i.e., resorbing) surface. This creates an enclosed extracellular compartment on the bone surface that is acidified by proton pumps in the ruffled membrane, and into which the osteoclast secretes proteolytic enzymes. The low pH of the compartment dissolves hydroxyapatite crystals at the bone surface, while the proteolytic enzymes digest the protein matrix. In this way, a resorption lacuna, or pit, is formed. At the end of this phase of the cycle, osteoblasts lay down a new protein matrix that is subsequently mineralized. In several disease states, such as osteoporosis and Paget's disease, the normal balance between bone resorption and formation is disrupted, and there is a net loss of bone at each cycle. Ultimately, this leads to weakening of the bone and may result in increased fracture risk with minimal trauma.

Several published studies have demonstrated that inhibitors of cysteine proteases are effective at inhibiting osteoclast-mediated bone resorption, and indicate an essential role for a cysteine proteases in bone resorption. For example, Delaisse, et al., Biochem. J, 1980, 192, 365, disclose a series of protease inhibitors in a mouse bone organ culture system and suggest that inhibitors of cysteine proteases (e.g., leupeptin, Z-Phe-Ala-CHN₂) prevent bone resorption, while serine protease inhibitors were ineffective. Delaisse, et al., Biochem. Biophys. Res. Commun., 1984, 125, 441, disclose that E-64 and leupeptin are also effective at preventing bone resorption in vivo, as measured by acute changes in serum calcium in rats on calcium deficient diets. Lerner, et al., J. Bone Min. Res., 1992, 7, 433, disclose that cystatin, an endogenous cysteine protease inhibitor, inhibits PTH stimulated bone resorption in mouse calvariae. Other studies, such as by Delaisse, et al., Bone, 1987, 8, 305, Hill, et al., J. Cell. Biochem., 1994, 56, 118, and Everts, et al., J. Cell. Physiol., 1992, 150, 221, also report a correlation between inhibition of cysteine protease activity and bone resorption. Tezuka, et al., J. Biol. Chem., 1994, 269, 1106, Inaoka, et al., Biochem. Biophys. Res. Commun., 1995, 206, 89 and Shi, et al., FEBS Lett., 1995, 357, 129 disclose that under normal conditions cathepsin K, a cysteine protease, is abundantly expressed in osteoclasts and may be the major cysteine protease present in these cells.

The abundant selective expression of cathepsin K in osteoclasts strongly suggests that this enzyme is essential for bone resorption. Thus, selective inhibition of cathepsin K may provide an effective treatment for diseases of excessive bone loss, including, but not limited to, osteoporosis, gingival diseases such as gingivitis and periodontitis, Paget's disease, hypercalcemia of malignancy, and metabolic bone disease. Cathepsin K levels have also been demonstrated to be elevated in chondroclasts of osteoarthritic synovium. Thus, selective inhibition of cathepsin K may also be useful for treating diseases of excessive cartilage or matrix degradation, including, but not limited to, osteoarthritis and rheumatoid arthritis. Metastatic neoplastic cells also typically express high levels of proteolytic enzymes that degrade the surrounding matrix. Thus, selective inhibition of cathepsin K may also be useful for treating certain neoplastic diseases.

Several cysteine protease inhibitors are known. Palmer, (1995) J. Med. Chem., 38, 3193, disclose certain vinyl sulfones which irreversibly inhibit cysteine proteases, such as the cathepsins B, L, S, O2 and cruzain. Other classes of compounds, such as aldehydes, nitriles, α-ketocarbonyl compounds, halomethyl ketones, diazomethyl ketones, (acyloxy)methyl ketones, ketomethylsulfonium salts and epoxy succinyl compounds have also been reported to inhibit cysteine proteases. See Palmer, id, and references cited therein.

U.S. Pat. No. 4,518,528 discloses peptidyl fluoromethyl ketones as irreversible inhibitors of cysteine protease. Published International Patent Application No. WO 94/04172, and European Patent Application Nos. EP 0 525 420 A1, EP 0 603 873 A1, and EP 0 611 756 A2 describe alkoxymethyl and mercaptomethyl ketones which inhibit the cysteine proteases cathepsins B, H and L. International Patent Application No. PCT/US94/08868 and and European Patent Application No. EP 0 623 592 A1 describe alkoxymethyl and mercaptomethyl ketones which inhibit the cysteine protease IL-1βconvertase. Alkoxymethyl and mercaptomethyl ketones have also been described as inhibitors of the serine protease kininogenase (International Patent Application No. PCT/GB91/01479).

Azapeptides which are designed to deliver the azaamino acid to the active site of serine proteases, and which possess a good leaving group, are disclosed by Elmore et al., Biochem. J., 1968, 107, 103, Garker et al., Biochem. J., 1974, 139, 555, Gray et al., Tetrahedron, 1977, 33, 837, Gupton et al., J. Biol. Chem., 1984, 259, 4279, Powers et al., J. Biol. Chem., 1984, 259, 4288, and are known to inhibit serine proteases. In addition, J. Med. Chem., 1992, 35, 4279, discloses certain azapeptide esters as cysteine protease inhibitors.

Antipain and leupeptin are described as reversible inhibitors of cysteine protease in McConnell et al., J. Med. Chem., 33, 86; and also have been disclosed as inhibitors of serine protease in Umezawa et al., 45 Meth. Enzymol. 678. E64 and its synthetic analogs are also well-known cysteine protease inhibitors (Barrett, Biochem. J., 201, 189, and Grinde, Biochem. Biophys. Acta, 701, 328).

1,3-diamido-propanones have been described as analgesic agents in U.S. Pat. Nos. 4,749,792 and 4,638,010.

EP 1 008 592 A2 describes cyclic amide derivatives which inhibit cathepsin K.

Thus, a structurally diverse variety of protease inhibitors have been identified. However, these known inhibitors are not considered suitable for use as therapeutic agents in animals, especially humans, because they suffer from various shortcomings. These shortcomings include lack of selectivity, cytotoxicity, poor solubility, and overly rapid plasma clearance. A need therefore exists for methods of treating diseases caused by pathological levels of proteases, particularly cysteine proteases, more particularly cathepsins, most particularly cathepsin K, and for novel inhibitor compounds useful in such methods.

We have now discovered a novel class of 4-amino-azepan-3-one compounds which are protease inhibitors, most particularly of cathepsin K.

### SUMMARY OF THE INVENTION

An object of the present invention is to provide 4-amino-azepan-3-one carbonyl protease inhibitors, particularly such inhibitors of cysteine and serine proteases, more particularly such compounds which inhibit cysteine proteases, even more particularly such compounds which inhibit cysteine proteases of the papain superfamily, yet more particularly such compounds which inhibit cysteine proteases of the cathepsin family, most particularly such compounds which inhibit cathepsin K, and which are useful for treating diseases which may be therapeutically modified by altering the activity of such proteases.

Accordingly, in the first aspect, this invention provides a compound according to Formula I.

In another aspect, this invention provides a pharmaceutical composition comprising a compound according to Formula I and a pharmaceutically acceptable carrier, diluent or excipient.

In yet another aspect, this invention provides intermediates useful in the preparation of the compounds of Formula I.

In still another aspect, this invention provides a method of treating diseases in which the disease pathology may be therapeutically modified by inhibiting proteases, particularly cysteine and serine proteases, more particularly cysteine proteases, even more particularly cysteine proteases of the papain superfamily, yet more particularly cysteine proteases of the cathepsin family, most particularly cathepsin K.

In a particular aspect, the compounds of this invention are especially useful for treating diseases characterized by bone loss, such as osteoporosis and gingival diseases, such as gingivitis and periodontitis, or by excessive cartilage or matrix degradation, such as osteoarthritis and rheumatoid arthritis.

### DETAILED DESCRIPTION OF THE INVENTION

The present invention provides compounds of Formula I:

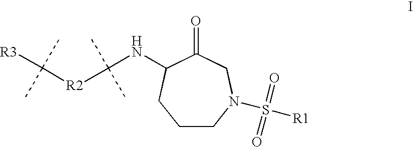

wherein:

R¹ is 2-pyridinyl;

R² is selected from the group consisting of: L-t-butyl-alaninyl, L-2-thiophenyl-alaninyl, L-cyclohexyl-glycinyl, L-allo-isoleucinyl, 1,2,3,4-tetrahydro-isoquinoline-3-carbonyl, L-prolinyl, (S)-2-amino-4-methanesulfonyl-butanoyl, and (S)-piperidine-2-carbonyl;

R³ is selected from the group consisting of: 3-methyl-benzofuran-2-carbonyl, benzofuran-2-carbonyl, 5-methoxy-benzofuran-2-carbonyl, benzo[b]thiophene-2-carbonyl, quinoline-2-carbonyl, quinoline-3-carbonyl, thiophene-2-carbonyl, thiophene-3-carbonyl, 5-methylthiophene-2-carbonyl, furan-2-carbonyl, furan-3-carbonyl, and thieno-[3,2-β]-thiophene-2-carbonyl;

and pharmaceutically acceptable salts, hydrates and solvates thereof.

The following compounds of Formula I are particularly preferred embodiments of the present invention:

quinoline-2-carboxylic acid {(S)-3,3-dimethyl-1-[(S)-3-oxo-1-(pyridine-2-sulfonyl)-azepan-4-ylcarbamoyl]-butyl}-amide (Example 1);

benzofuran-2-carboxylic acid {(S)-3,3-dimethyl-1-[(S)-3-oxo-1-(pyridine-2-sulfonyl)-azepan-4-ylcarbamoyl]-butyl}-amide (Example 2);

5-methoxy-benzofuran-2-carboxylic acid {(S)-3,3-dimethyl-1-[(S)-3-oxo-1-(pyridine-2-sulfonyl)-azepan-4-ylcarbamoyl]-butyl}-amide (Example 3);

benzo[b]thiophene-2-carboxylic acid {(S)-3,3-dimethyl-1-[(R)-3-oxo-1-(pyridine-2-sulfonyl)-azepan-4-ylcarbamoyl]-butyl}-amide (Example 4);

thiophene-2-carboxylic acid {(S)-3,3-dimethyl-1-[(S)-3-oxo-1-(pyridine-2-sulfonyl)-azepan-4-ylcarbamoyl]-butyl}-amide (Example 7);

5-methyl-thiophene-2-carboxylic acid {(S)-3,3-dimethyl-1-[(S)-3-oxo-1-(pyridine-2-sulfonyl)-azepan-4-ylcarbamoyl]-butyl}-amide (Example 9);

furan-2-carboxylic acid {(S)-3,3-dimethyl-1-[(S)-3-oxo-1-(pyridine-2-sulfonyl)-azepan-4-ylcarbamoyl]-butyl}-amide (Example 10);

furan-3-carboxylic acid {(S)-3,3-dimethyl-1-[(S)-3-oxo-1-(pyridine-2-sulfonyl)-azepan-4-ylcarbamoyl]-butyl}-amide (Example 11);

thieno[3,2-b]thiophene-2-carboxylic acid {(S)-3,3-dimethyl-1-[(S)-3-oxo-1-(pyridine-2-sulfonyl)-azepan-4-ylcarbamoyl]-butyl}-amide (Example 12);

thieno[3,2-b]thiophene-2-carboxylic acid {(S)-1-[(S)-3-oxo-1-(pyridine-2-sulfonyl)-azepan-4-ylcarbamoyl]-2-thiophen-2-yl-ethyl}-amide (Example 24);

thieno[3,2-b]thiophene-2-carboxylic acid {(S)-1-cyclohexyl-1-[(S)-3-oxo-1-(pyridine-2-sulfonyl)-azepan4-ylcarbamoyl]-methyl}-amide (Example 36); and

thieno[3,2-b]thiophene-2-carboxylic acid {(1S,2R)-2-methyl-1-[(S)-3-oxo-1-(pyridine-2-sulfonyl)-azepan-4-ylcarbamoyl]-butyl}-amide (Example 48).

A specific representative compound of the present invention is set forth in Example 1.

Compared to the corresponding 5 and 6 membered ring compounds, the 7 membered ring compounds of the present invention are configurationally more stable at the carbon center alpha to the ketone.

The present invention includes deuterated analogs of the inventive compounds. The deuterated compounds of the present invention should exhibit superior chiral stability compared to the protonated isomer.

Where possible the present invention includes quaternary salts of the inventive compounds.

### Definitions

The present invention includes all hydrates, solvates, complexes and prodrugs of the compounds of this invention. Prodrugs are any covalently bonded compounds which release the active parent drug according to Formula I in vivo. If a chiral center or another form of an isomeric center is present in a compound of the present invention, all forms of such isomer or isomers, including enantiomers and diastereomers, are intended to be covered herein. Inventive compounds containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form.

The meaning of any substituent at any one occurrence in Formula I or any subformula thereof is independent of its meaning, or any other substituent's meaning, at any other occurrence, unless specified otherwise.

Abbreviations and symbols commonly used in the peptide and chemical arts are used herein to describe the compounds of the present invention. In general, the amino acid abbreviations follow the IUPAC-IUB Joint Commission on Biochemical Nomenclature as described in Eur. J. Biochem., 158, 9 (1984).

“Proteases” are enzymes that catalyze the cleavage of amide bonds of peptides and proteins by nucleophilic substitution at the amide bond, ultimately resulting in hydrolysis. Such proteases include: cysteine proteases, serine proteases, aspartic proteases, and metalloproteases. The compounds of the present invention are capable of binding more strongly to the enzyme than the substrate and in general are not subject to cleavage after enzyme catalyzed attack by the nucleophile. They therefore competitively prevent proteases from recognizing and hydrolyzing natural substrates and thereby act as inhibitors.

The term “amino acid” as used herein refers to the D- or L-isomers of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine.

A representation of an element is understood to include all isotopes of that element. Thus, for example, the term “H” includes all isotopes of hydrogen, including deuterium.

Here and throughout this application the term C₀ denotes the absence of the substituent group immediately following; for instance, in the moiety ArC₀₋₆alkyl, when C is 0, the substituent is Ar, e.g., phenyl. Conversely, when the moiety ArC₀₋₆alkyl is identified as a specific aromatic group, e.g., phenyl, it is understood that the value of C is 0.

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical.

Certain reagents are abbreviated herein. DMF refers to dimethyl formamide, TEA refers to triethylamine, NMM refers to N-methylmorpholine, TFA refers to trifluoroacetic acid, and TMSOTf refers to trimethylsilyl trifluoromethanesulfonate.

### Methods of Preparation

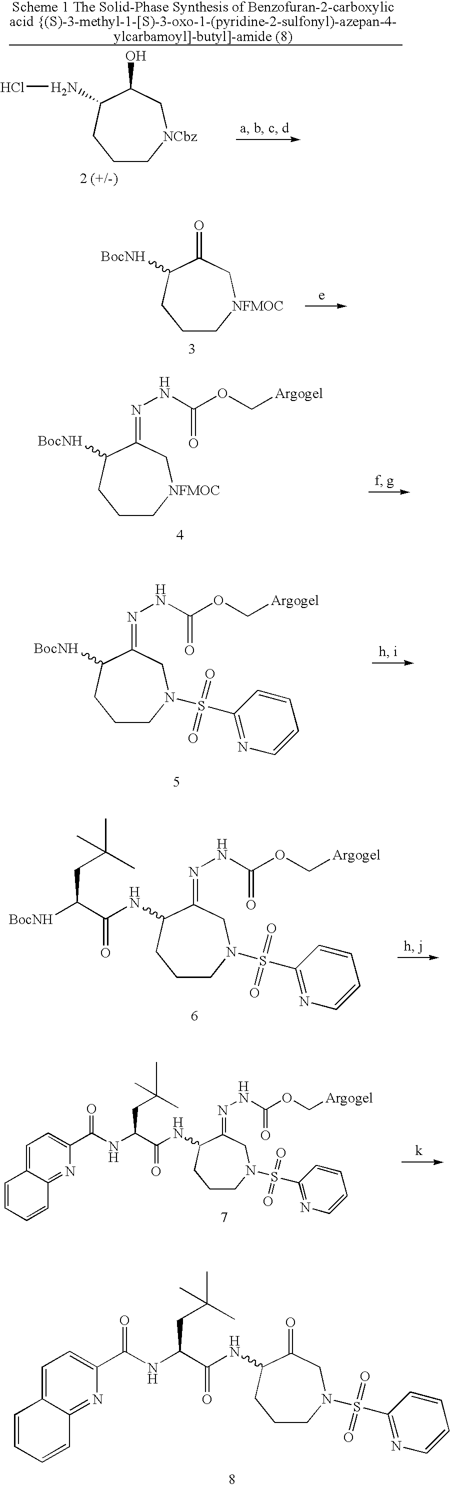

Boc=tBuOCO; FMOC=9H-fluoren-9-ylmethyl-OCO; Argogel=polyethylene glycol-polystyrene co-polymeric beads; a: tBuOCOCO₂tBu, Et₃N, CH₂Cl₂; b: H₂, Pd/C, EtOH; c: FMOC-N-hydroxy-succinimide, Et₃N, CH₂Cl₂; d: Dess-Martin periodinane, CH₂Cl₂; e: Argogel-CONHNH₂, Et₃N, CH₂Cl₂; f: 20% piperidine, DMF; g: Phenyl-SO₂Cl, NMM, DMF; h: TMSOTf, 2,6-lutidine; CH₂Cl₂; i: Boc-L-t-butylalanine-OH, EDC, NMM, DMF; j: 2-quinoline-CO₂H, EDC, TEA, DMF; k: TFA, MeCHO, H₂O, CF₃CH₂OH

Azapan-3-ol (2, Marquis, R. et al J. Med. Chem. 2001)) was protected as a t-butyloxycarbamate (Boc) using di-t-butyl-dicarbonate; the Cbz group was deprotected by hydrogenolysis, then the secondary amine was reprotected with FMOC using 9-fluorenylmethyl carbonyl-N-hydroxy-succinimide. The alcohol was oxidized to ketone 3 using Dess-Martin periodinane (Dess, D. B.; Martin, J. C. J. Org. Chem. 1983, 48, 4155-4156). Ketone 3 was converted to the immobilized hydrazone 4 using a hydrazide carbamate covalently linked to Argogel beads (a polyethyleneglycol/polystyrene copolymer that has excellent swelling properties in a variety of solvents) using a procedure described by Lee, A.; Huang, L.; Ellman, J. A in J. Am. Chem. Soc. 1999, 121, 9907-9914. Next, the FMOC group was deprotected to provide the free secondary amine using standard conditions of piperidine in DMF. Treatment with phenylsulfonyl chloride gave the desired sulfonamide 5. The Boc group was deprotected with trimethylsilyltriflate and 2,6-lutidine employing the methodology as described in Zhang, A. J.; Russell, D. H.; Zhu, J.; Burgess, K. Tet Lett. 1998, 39, 7439-7442. To complete the synthesis, Boc-L-phenylalanine was coupled using standard peptide coupling conditions with 1-(3-dimethylaminopropyl)-3-ethylcarbodiimide hydrochloride (EDC) and N-methylmorpholine in DMF to provide amide 6. Then, the Boc-deprotection conditions using trimethylsilyl-triflate were employed again to selectively free the primary amine of the phenylalaninamide without cleavage of the hydrazone linker. Acylation of the amine was accomplished using standard peptide coupling conditions of EDC and triethylamine in DMF to provide amide 7. Finally, cleavage of the hydrazone using previously described optimal cleavage conditions (1:4:4:15 trifluoroacetic acid, water, acetaldehyde, and 2,2,2-trifluoroethanol, by Lee, A.; Huang, L.; Ellman, J. A in J. Am. Chem. Soc. 1999, 121, 9907-9914) afforded the desired azepanone 8 as a 1:1 mixture of epimers.

The starting materials used herein are commercially available amino acids or are prepared by routine methods well known to those of ordinary skill in the art and can be found in standard reference books, such as the COMPENDIUM OF ORGANIC SYNTHETIC METHODS, Vol. I-VI (published by Wiley-Interscience).

Coupling methods to form amide bonds herein are generally well known to the art. The methods of peptide synthesis generally set forth by Bodansky et al., THE PRACTICE OF PEPTIDE SYNTHESIS, Springer-Verlag, Berlin, 1984; E. Gross and J. Meienhofer, THE PEPTIDES, Vol. 1, 1-284 (1979); and J. M. Stewart and J. D. Young, SOLID PHASE PEPTIDE SYNTHESIS, 2d Ed., Pierce Chemical Co., Rockford, Ill., 1984. are generally illustrative of the technique and are incorporated herein by reference.

Synthetic methods to prepare the compounds of this invention frequently employ protective groups to mask a reactive functionality or minimize unwanted side reactions. Such protective groups are described generally in Green, T. W, PROTECTIVE GROUPS IN ORGANIC SYNTHESIS, John Wiley & Sons, New York (1981). The term “amino protecting groups” generally refers to the Boc, acetyl, benzoyl, Fmoc and Cbz groups and derivatives thereof as known to the art. Methods for protection and deprotection, and replacement of an amino protecting group with another moiety are well known.

Acid addition salts of the compounds of Formula I are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. Certain of the compounds form inner salts or zwitterions which may be acceptable. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are specific examples of cations present in pharmaceutically acceptable salts. Halides, sulfate, phosphate, alkanoates (such as acetate and trifluoroacetate), benzoates, and sulfonates (such as mesylate) are examples of anions present in pharmaceutically acceptable salts. Quaternary ammonium salts are prepared by treating a parent amine compound with an excess of alkyl halide, such as methyl iodide.

This invention also provides a pharmaceutical composition which comprises a compound according to Formula I and a pharmaceutically acceptable carrier, diluent or excipient. Accordingly, the compounds of Formula I may be used in the manufacture of a medicament. Pharmaceutical compositions of the compounds of Formula I prepared as hereinbefore described may be formulated as solutions or lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride or sodium citrate.

Alternately, these compounds may be encapsulated, tableted or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

For rectal administration, the compounds of this invention may also be combined with excipients such as cocoa butter, glycerin, gelatin or polyethylene glycols and molded into a suppository.

### Utility of the Present Invention

The compounds of Formula I are useful as protease inhibitors, particularly as inhibitors of cysteine and serine proteases, more particularly as inhibitors of cysteine proteases, even more particularly as inhibitors of cysteine proteases of the papain superfamily, yet more particularly as inhibitors of cysteine proteases of the cathepsin family, most particularly as inhibitors of cathepsin K. The present invention also provides useful compositions and formulations of said compounds, including pharmaceutical compositions and formulations of said compounds.

The present compounds are useful for treating diseases in which cysteine proteases are implicated, including infections by pneumocystis carinii, trypsanoma cruzi, trypsanoma brucei, and Crithidia fusiculata; as well as in schistosomiasis, malaria, tumor metastasis, metachromatic leukodystrophy, muscular dystrophy, amytrophy; and especially diseases in which cathepsin K is implicated, most particularly diseases of excessive bone or cartilage loss, including osteoporosis, gingival disease including gingivitis and periodontitis, arthritis, more specifically, osteoarthritis and rheumatoid arthritis, Paget's disease; hypercalcemia of malignancy, and metabolic bone disease.

Metastatic neoplastic cells also typically express high levels of proteolytic enzymes that degrade the surrounding matrix, and certain tumors and metastatic neoplasias may be effectively treated with the compounds of this invention.

The present invention also provides methods of treatment of diseases caused by pathological levels of proteases, particularly cysteine and serine proteases, more particularly cysteine proteases, even more particularly cysteine proteases of the papain superfamily, yet more particularly cysteine proteases of the cathepsin family, which methods comprise administering to an animal, particularly a mammal, most particularly a human in need thereof a compound of the present invention. The present invention especially provides methods of treatment of diseases caused by pathological levels of cathepsin K, which methods comprise administering to an animal, particularly a mammal, most particularly a human in need thereof an inhibitor of cathepsin K, including a compound of the present invention. The present invention particularly provides methods for treating diseases in which cysteine proteases are implicated, including infections by pneumocystis carinii, trypsanoma cruzi, trypsanoma brucei, and Crithidia fusiculata; as well as in schistosomiasis, malaria, tumor metastasis, metachromatic leukodystrophy, muscular dystrophy, amytrophy, and especially diseases in which cathepsin K is implicated, most particularly diseases of excessive bone or cartilage loss, including osteoporosis, gingival disease including gingivitis and periodontitis, arthritis, more specifically, osteoarthritis and rheumatoid arthritis, Paget's disease, hypercalcemia of malignancy, and metabolic bone disease.

This invention further provides a method for treating osteoporosis or inhibiting bone loss which comprises internal administration to a patient of an effective amount of a compound of Formula I, alone or in combination with other inhibitors of bone resorption, such as bisphosphonates (i.e., allendronate), hormone replacement therapy, anti-estrogens, or calcitonin. In addition, treatment with a compound of this invention and an anabolic agent, such as bone morphogenic protein, iproflavone, may be used to prevent bone loss or to increase bone mass.

For acute therapy, parenteral administration of a compound of Formula I is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit cathepsin K. The compounds are administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to inhibit bone resorption or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of the present invention are administered in accordance with the present invention.

### Biological Assays

The compounds of this invention may be tested in one of several biological assays to determine the concentration of compound which is required to have a given pharmacological effect.

Determination of Cathepsin K Proteolytic Catalytic Activity

All assays for cathepsin K were carried out with human recombinant enzyme. Standard assay conditions for the determination of kinetic constants used a fluorogenic peptide substrate, typically Cbz-Phe-Arg-AMC, and were determined in 100 mM Na acetate at pH 5.5 containing 20 mM cysteine and 5 mM EDTA. Stock substrate solutions were prepared at concentrations of 10 or 20 mM in DMSO with 20 uM final substrate concentration in the assays. All assays contained 10% DMSO. Independent experiments found that this level of DMSO had no effect on enzyme activity or kinetic constants. All assays were conducted at ambient temperature. Product fluorescence (excitation at 360 nM; emission at 460 nM) was monitored with a Perceptive Biosystems Cytofluor II fluorescent plate reader. Product progress curves were generated over 20 to 30 minutes following formation of AMC product.

Inhibition Studies

Potential inhibitors were evaluated using the progress curve method. Assays were carried out in the presence of variable concentrations of test compound. Reactions were initiated by addition of enzyme to buffered solutions of inhibitor and substrate. Data analysis was conducted according to one of two procedures depending on the appearance of the progress curves in the presence of inhibitors. For those compounds whose progress curves were linear, apparent inhibition constants (K_{i,app}) were calculated according to equation 1 (Brandt et al., Biochemitsry, 1989, 28, 140):

v=VₘA/[Kₐ(1+I/K_{i, app})+A] (1)

where v is the velocity of the reaction with maximal velocity Vₘ, A is the concentration of substrate with Michaelis constant of Kₐ, and I is the concentration of inhibitor.

For those compounds whose progress curves showed downward curvature characteristic of time-dependent inhibition, the data from individual sets was analyzed to give k_{obs} according to equation 2:

[AMC]=vₛₛt+(v₀−vₛₛ) [1−exp(−k_{obs}t)]/k_{obs} (2)

where [AMC] is the concentration of product formed over time t, v₀ is the initial reaction velocity and vₛₛ is the final steady state rate. Values for k_{obs} were then analyzed as a linear function of inhibitor concentration to generate an apparent second order rate constant (k_{obs}/inhibitor concentration or k_{obs}/[I]) describing the time-dependent inhibition. A complete discussion of this kinetic treatment has been fully described (Morrison et al., Adv. Enzymol. Relat. Areas Mol. Biol., 1988, 61, 201).

This assay measures the affinity of inhibitors to cysteine proteases, in this case, cathepsin K. The skilled artisan would consider any compound exhibiting a Kᵢ value of less than 50 micromolar, preferably less than 1 micromolar, to be a potential lead compound for further research. Most preferable are compounds exhibiting a Kᵢ of less than 100 nM. The skilled artisan would consider such compound to be a drug development drug candidate assuming an acceptable pathology/toxicology profile and in vivo activity.

The Ki values for compounds of the present invention range from 2 nM to >1000 nM against cathepsin K.

Human Osteoclast Resorption Assay

Aliquots of osteoclastoma-derived cell suspensions were removed from liquid nitrogen storage, warmed rapidly at 37° C. and washed ×1 in RPMI-1640 medium by centrifugation (1000 rpm, 5 min at 4° C.). The medium was aspirated and replaced with murine anti-HLA-DR antibody, diluted 1:3 in RPM[-1640 medium, and incubated for 30 min on ice. The cell suspension was mixed frequently.

The cells were washed ×2 with cold RPMI-1640 by centrifugation (1000 rpm, 5 min at 4° C.) and then transferred to a sterile 15 mL centrifuge tube. The number of mononuclear cells were enumerated in an improved Neubauer counting chamber.

Sufficient magnetic beads (5/mononuclear cell), coated with goat anti-mouse IgG, were removed from their stock bottle and placed into 5 mL of fresh medium (this washes away the toxic azide preservative). The medium was removed by immobilizing the beads on a magnet and is replaced with fresh medium.

The beads were mixed with the cells and the suspension was incubated for 30 min on ice. The suspension was mixed frequently. The bead-coated cells were immobilized on a magnet and the remaining cells (osteoclast-rich fraction) were decanted into a sterile 50 mL centrifuge tube. Fresh medium was added to the bead-coated cells to dislodge any trapped osteoclasts. This wash process was repeated ×10. The bead-coated cells were discarded.

The osteoclasts were enumerated in a counting chamber, using a large-bore disposable plastic pasteur pipette to charge the chamber with the sample. The cells were pelleted by centrifugation and the density of osteoclasts adjusted to 1.5×10⁴/mL in EMEM medium, supplemented with 10% fetal calf serum and 1.7 g/litre of sodium bicarbonate. 3 mL aliquots of the cell suspension ( per treatment) were decanted into 15 mL centrifuge tubes. These cells were pelleted by centrifugation. To each tube 3 mL of the appropriate treatment was added (diluted to 50 uM in the EMEM medium). Also included were appropriate vehicle controls, a positive control (87MEM1 diluted to 100 ug/mL) and an isotype control (IgG2a diluted to 100 ug/mL). The tubes were incubated at 37° C. for 30 min.

0.5 mL aliquots of the cells were seeded onto sterile dentine slices in a 48-well plate and incubated at 37° C. for 2 h. Each treatment was screened in quadruplicate. The slices were washed in six changes of warm PBS (10 mL/well in a 6-well plate) and then placed into fresh treatment or control and incubated at 37° C. for 48 h. The slices were then washed in phosphate buffered saline and fixed in 2% glutaraldehyde (in 0.2M sodium cacodylate) for 5 min., following which they were washed in water and incubated in buffer for 5 min at 37° C. The slices were then washed in cold water and incubated in cold acetate buffer/fast red garnet for 5 min at 4° C. Excess buffer was aspirated, and the slices were air dried following a wash in water.

The TRAP positive osteoclasts were enumerated by bright-field microscopy and were then removed from the surface of the dentine by sonication. Pit volumes were determined using the Nikon/Lasertec ILM21W confocal microscope.

### General

Amino acid derivatives were purchased from Bachem or Novabiochem Intl. Di-t-butyl-dicarbonate, triethylamine, carboxylic acids, piperidine, EDC, NMM, DMF (99.8%), methylene chloride, acetaldehyde, 2,2,2-trifluoroethanol, 2-mercaptopyridine, ethanol, hydrazine hydrate, triphosgene, TFA, TMSOTf, and FMOC chloride, and a chlorine gas cylinder were purchased from Aldrich Chemical Co., Inc. A hydrogen gas cylinder was purchased from Praxair. Dess-Martin periodinane was purchased from Albany Science. Argogel resin (lot no. 00178; P/N 800004) was purchased from Argonaut Technologies. All solvents were HPLC grade and used as purchased without purification. Radiofrequncy encoding/sorting equipment was purchased from IRORI, a Discovery Partners International Company. All reactions described in Scheme 3 were carried out in IRORI MiniKan reactors initially filled with 70 mg of hydrazinecarboxylic acid (polyethyleneglycol-polystyrene copolymer) ester (derived from Argogel beads carbonyl diimidazaole and hydrazine) resin. Purities of final cleavage products were estimated by LC-MS. Representative compounds cleaved from solid support were characterized by ¹H NMR and LCMS. ¹H NMR spectra were obtained and recorded on Varian 300 spectrometer and were calibrated using residual undeuterated solvent as an internal reference.

CDCl₃ is deuteriochloroform. Chemical shifts are reported in parts per million (d) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. Mass spectra were taken on either VG 70 FE, PE Syx API III, or VG ZAB HF instruments, using fast atom bombardment (FAB) or electrospray (ES) ionization techniques.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel.

HPLC was conducted using a 20 mm×50 mm YMC reversed-phase column on Gilson 215.

### EXAMPLES

In the following synthetic examples, temperature is in degrees Centigrade (° C.). Unless otherwise indicated, all of the starting materials were obtained from commercial sources. Without further elaboration, it is believed that one skilled in the art can, using the preceding description, utilize the present invention to its fullest extent. These Examples are given to illustrate the invention, not to limit its scope. Reference is made to the claims for what is reserved to the inventors hereunder.

### Example 1

### Quinoline-2-carboxylic acid {(S)-3,3-dimethyl-1-[(S)-3-oxo-1-(pyridine-2-sulfonyl)-azepan-4-ylcarbamoyl]-butyl}-amide

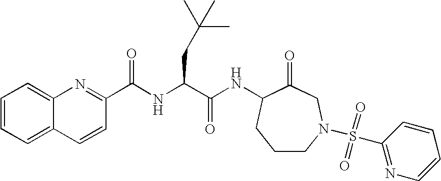

### 4-Boc-amino-3-hydroxy-azepane-1-carboxylic acid benzyl ester

4-Amino-3-hydroxy-azepane-1-carboxylic acid benzyl ester hydrochloride (2, 25.0 g, 83 mmol) was dissolved in 1,4-dioxane (250 ml), then sodium hydroxide (6.64 g, 166 mmol) in water (50 ml) was added dropwise at 0° C. Di-t-butyl-dicarbonate (20 g, 92 mmol) was added in one portion, then the reaction mixture was warmed to RT overnight. The reaction mixture was then concentrated by rotary evaporation, then the resulting residue was dissolved in ethyl acetate, extracted with water, then 10% aqueous NaHCO₃, then 1 N aq. HCl, then water, then brine. The combined organics were dried with magnesium sulfate, filtered, concentrated by rotary evaporation, and yielded a white solid (33 g, >theor. yield) that was used in the next reaction without further purification: ¹H NMR(CDCl₃) 7.39-7.29 (s, 5H); 5.23-5.12(m, 2H); 4.73-4.63(m, 1H); 3.89-4.63(m, 7H); 2.07-1.80(m,4H); 1.26(s, 9H), MS: M+H=365.2

### (3-Hydroxy-azepan-4-yl)-carbamic acid-tert-butyl ester

4-Boc-amino-3-hydroxy-azepane-1-carboxylic acid benzyl ester (20 g, 54.0 mmol) was dissolved in ethanol (250 ml). The solution was degassed by bubbling argon gas for 5 minutes. Then 10% Pd/C (5 g) was added and the solution was attached to a Paar hydrogenator and was stirred under hydrogen gas for 6 h at RT. The reaction mixture was filtered through Celite, then concentrated by rotary evaporation to yield a white solid (12 g, 97% yield) and was used in the next reaction without further purification: ¹H NMR (CDCl3, 400 MHz) 4.80(br, 1H); 3.76-3.50(m, 2H); 3.05-2.81(m, 6H); 1.89-1.53(m, 4H); 1.45(s,9H), MS: M+H=231.2

### 4Boc-amino-3-hydroxy-azepane-1-carboxylic acid 9H-fluoren-9-ylmethyl ester

Sodium bicarbonate (8.1 g, 96 mmol) was added, then 9-fluorenylmethyl carbonyl-N-hydroxy-succinimide (29.7 g, 88 mmol) was added to a mixture of (3-Hydroxy-azepan-4-yl)-carbamic acid-tert-butyl ester (18.5 g, 80.4 mmol) in acetone (300 ml) and water (300 ml). The reaction mixture was stirred at RT for 1 h. The acetone was removed by rotary evaporation, and the aqueous layer was extracted repeatedly with ethyl acetate. The combined organics were then extracted with 10% sodium bicarbonate, then brine. The combined organics were dried with magnesium sulfate, filtered, concentrated by rotary evaporation, and purified by flash column chromatography (0 to 2% MeOH/CH₂Cl₂) to give the desired product as a white solid (31 g, 85% yield): ¹H NMR (CDCl3, 400 MHz) 8.00-7.28(m, 9H); 4.75-4.50(m, 4H); 3.90-3.00(m, 7H); 1.90-1.30(m, 12H), MS: M+H=453.2

### 4-Boc-amino-3-oxo-azepane-1-carboxylic acid 9H-fluoren-9-ylmethyl ester (3)

Dess-Martin periodinane (25 g, 59 mmol) was added to a solution of 4-Boc-amino-3-hydroxy-azepane-1-carboxylic acid 9H-fluoren-9-ylmethyl ester (24.2 g, 53.6 mmol) was dissoved in CH₂Cl₂ (500 ml) and the reaction was stirred for 2 h at RT. The reaction mixture was then extracted with 10% sodium bicarbonate, 10% sodium bisulfite, then brine. The combined organics were dried with magnesium sulfate, filtered, concentrated by rotary evaporation, and purified by flash column chromatography (20% EtOAc/hexanes) to give the desired product as a white solid (19.4 g, 80% yield): ¹H NMR (CDCl₃, 400 MHz) 8.00-7.28(m, 9H); 4.75-3.60(m, 11H); 2.90-1.30(m, 10H), MS: M+H=451.2

### 4-Boc-amino-3-(polyethylene glycol-polystyrene co-polymer-carbonyl-hydrazono)-azepane-1-carboxylic acid 9H-fluoren-9-ylmethyl ester (4)

Hydrazinecarboxylic acid (polyethyleneglycol-polystyrene co-polymer) ester (derived from Argogel beads carbonyl diimidazaole and hydrazine, ¹⁶70 mg, 0.028 mmol) was added to an IRORI MicroKan, then was immersed in a solution containg 4-Boc-amino-3-oxo-azepane-1-carboxylic acid 9H-fluoren-9-ylmethyl ester (7 equivalents, 0.8 M in THF), and the reaction was heated to 45° C. overnight. The reaction mixture was filtered, washed with THF, 1:1 MeOH/CH₂Cl₂, and dried in vacuo.

### N′-(4-Boc-amino-azepan-3-ylidene)-hydrazinecarboxylic acid (polyethylene glycol-polystyrene co-polymer) ester

4-Boc-amino-3-(polyethylene glycol-polystyrene co-polymer-carbonyl-hydrazono)-azepane-1-carboxylic acid 9H-fluoren-9-ylmethyl ester (0.028 mmol) in an IRORI MicroKan was added to a solution of 20% piperidine/DMF (500ml)and was shaken at RT for 1 h. The reaction mixture was filtered, washed repeatedly with THF and CH₂Cl₂, and MeOH, and the solid was dried under aspirator pressure.

### N′-[4-Boc-amino-1-(2-pyridine-sulfonyl)-azepan-3-ylidene]-hydrazinecarboxylic acid (polyethylene glycol-polystyrene co-polymer) ester

N′-(4-Boc-amino-azepan-3-ylidene)-hydrazinecarboxylic acid (polyethylene glycol-polystyrene co-polymer) ester (0.028 mmol) in an IPORI MicroKan was immersed in a solution of 2-pyridinesulfonyl chloride (10 equivalents, 0.1M) and NMM (12 equivalents, 0.12M) and the reaction was shaken at RT overnight, then filtered. The IRORI microKan was washed with THF and then CH₂Cl₂ and then MeOH, and the solid was dried under aspirator pressure.

### N′-[4-Amino-1-(2-pyridinesulfonyl)-azepan-3-ylidene]-hydrazinecarboxylic acid (polyethylene glycol-polystyrene co-polymer) ester

N′-[4-Boc-amino-1-(2-pyridine-sulfonyl)-azepan-3-ylidene]-hydrazinecarboxylic acid (polyethylene glycol-polystyrene co-polymer) ester (0.028 mmol) in an IRORI MicroKan was immersed in a solution of 2,6-lutidine (0.5M), trimethylsilyltriflate (1.5 M) in CH₂Cl₂, and the reaction was shaken at RT for 1 h, then filtered. Then, the resin was again immersed in a solution of 2,6-lutidine (0.35M), trimethylsilyltriflate (1.0 M) in CH₂Cl₂, and the reaction was shaken at RT for 2 h, then filtered. The reaction mixture was filtered, washed repeatedly with THE and CH₂Cl₂, and the solid was dried under aspirator pressure.

### N′-[4-{(S)-2-Boc-amino]-4,4-dimethyl-pentanoylamino}-1-(pyridine-2-sulfonyl)-azepan-3-ylidene]-hydrazinecarboxylic acid (polyethyleneglycol-polystyrene-copolymer) ester

N′-[4-Amino-1-(2-pyridinesulfonyl)-azepan-3-ylidene]-hydrazinecarboxylic acid (polyethylene glycol-polystyrene co-polymer) ester (0.028 mmol) in an IRORI MicroKlan was immersed Boc-L-t-butyl-alanine (5 equivalents, 0.07 M) and N-methyl morpholine (10 equivalents, 0.14 M) in DMF. Then, EDC (5 equivalents), HOBT (5 equivalents) was added and the reaction mixture was shaken at RT overnight. The reaction mixture was filtered, washed repeatedly with THF and CH₂Cl₂, and the solid was dried under aspirator pressure.

### N′-[4-{(S)-2-amino]-4,4-dimethyl-pentanoylamino}-1-(pyridine-2-sulfonyl)-azepan-3-ylidene]-hydrazinecarboxylic acid (polyethyleneglycol-polystyrene-copolymer) ester

N′-[4-{(S)-2-Boc-amino]-4,4-dimethyl-pentanoylamino}-1-(pyridine-2-sulfonyl)-azepan-3-ylidene]-hydrazinecarboxylic acid (polyethyleneglycol-polystyrene-copolymer) ester (0.028 mmol) in an IRORI MicroKan was immersed in a solution of 2,6-lutidine (0.5 M), trimethylsilyltriflate (1.5 M) in CH₂Cl₂, and the reaction was shaken at RT for 1 h, then filtered. Then, the resin was again immersed in a solution of 2,6-lutidine (0.35 M), trimethylsilyltriflate (1.0 M) in CH₂Cl₂, and the reaction was shaken at RT for 2 h, then filtered. The reaction mixture was filtered, washed repeatedly with THF and CH₂Cl₂, and the solid was dried under aspirator pressure.

### N′-[1-(2-pyridine-sulfonyl)-4-{(S)-2-[(1-quinoline-2-yl-methanoyl)-amino]-4,4-dimethylpentanoylamino}azepan-3-ylidene]-hydrazinecarboxylic acid (polyethyleneglycol-polystyrene-copolymer) ester

N′-[4-{(S)-2-amino]-4,4-dimethyl-pentanoylamino}-1-(pyridine-2-sulfonyl)-azepan-3-ylidene]-hydrazinecarboxylic acid (polyethyleneglycol-polystyrene-copolymer) ester (0.028 mmol) in an IRORI MicroKan was immersed in a solution of triethylamine (10 equivalents, 0.15 M), 2-quinaldic acid (5 equivalents, 0.075 M), and EDC (5 equivalents, 0.075 M), HOBT (5 equivalents, 0.075 M) in DMF, and the reaction was shaken at RT overnight. The reaction mixture was filtered, washed repeatedly with THF and CH₂Cl₂, and the solid was dried under aspirator pressure.

### Quinoline-2-carboxylic acid {(S)-3,3-dimethyl-1-[(S)-3-oxo-1-(pyridine-2-sulfonyl)-azepan-4-ylcarbamoyl]-butyl]-amide

N′-[1-(pyridine-2-sulfonyl)-4-{(S)-2-[(1-quinoline-2-yl-methanoyl)-amino]-4,4-dimethyl-pentanoylamino}-azepan-3-ylidene]-hydrazinecarboxylic acid (polyethyleneglycol-polystyrene-copolymer) ester (0.028 mmol) in an IRORI MicroKan was suspended in the standard cleavage conditions (3 ml, 1:4:4:15 trifluoroacetic acid, water, acetaldehyde, and 2,2,2-trifluoroethanol) and the reaction was shaken overnight and then treated with 2 ml cleavage solution for 1 hr. The reaction mixture was filtered and washed wih THF, CH₂Cl₂, and MeOH, and the combined solutions were collected and concentrated in vacuo. The crude reaction product was evaluated by LCMS: M+H⁺=552.4. The product was then purified by flash column chromatography and gave the desired product as a white solid (2.1 mg, 14% overall yield). H-NMR (400 MHz, CDCl₃) δ 8.72-8.60(m, 2H), 8.45-8.20(m, 3H), 8.00-7.20(m, 7H), 5.15(m, 1H), 4.77(m, 2H), 4.18(d, 1H), 3.85(t, 1H), 2.75 (t, 1H), 2.40-1.40 (m, 6H), 1.04 (s, 9H).

### Examples 2-90

The compounds of Examples 2-90 in Table I were made according to the synthesis of Scheme 1.

TABLE I

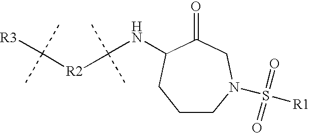

| Ex- | | | | | MS |
|---|---|---|---|---|---|
| am- | | | | | (ES+) |
| ple | R1 | R2 | R3 | Structure | m/e |
| 1. | 2-pyridinyl | L-t-butyl- alanine | quinoline- 2-carbonyl | 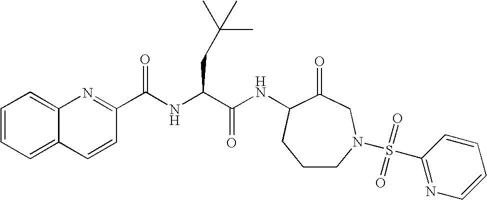 | (M + H) 552.4 |
| 2. | 2-pyridinyl | L-t-butyl- alanine | benzofuran- 2- carbonyl- | 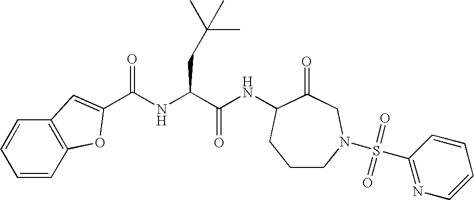 | (M + H) 541.4 |
| 3. | 2-pyridinyl | L-t-butyl- alanine | 5-methoxy- benzofuran- 2- carbonyl- | 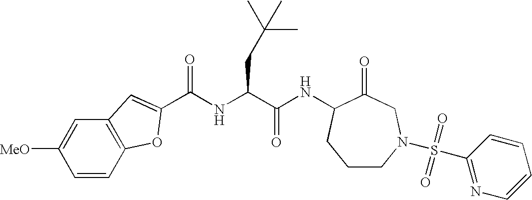 | (M + H) 571.4 |
| 4. | 2-pyridinyl | L-t-butyl- alanine | benzothio- phene-2- carbonyl- | 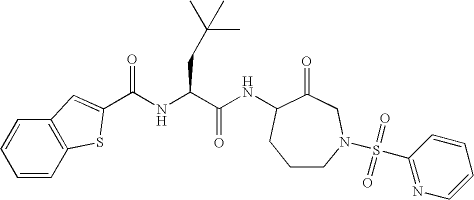 | (M + H) 557.2 |
| 5. | 2-pyridinyl | L-t-butyl- alanine | 3-methyl- benzofuran- 2- carbonyl- | 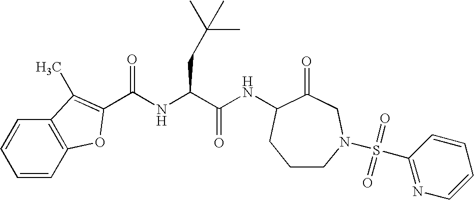 | (M + H) 555.2 |
| 6. | 2-pyridinyl | L-t-butyl- alanine | quinoline- 3-carbonyl | 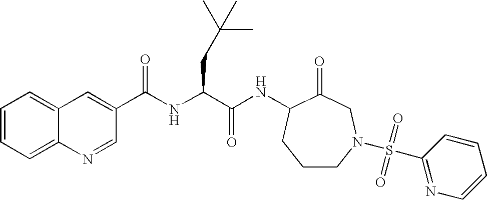 | (M + H) 552.4 |
| 7. | 2-pyridinyl | L-t-butyl- alanine | thiophene- 2-carbonyl | 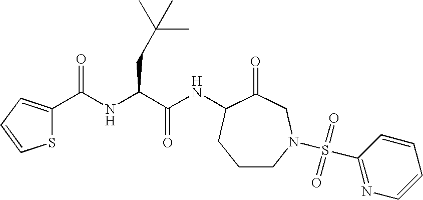 | (M + H) 507.2 |
| 8. | 2-pyridinyl | L-t-butyl- alanine | thiophene- 3-carbonyl | 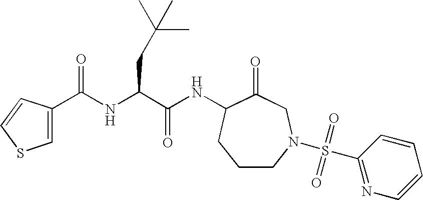 | (M + H) 507.2 |
| 9. | 2-pyridinyl | L-t-butyl- alanine | 5- methylthio phene-2- carbonyl | 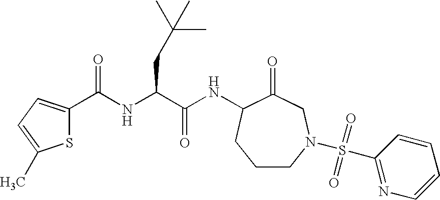 | (M + H) 521.4 |
| 10. | 2-pyridinyl | L-t-butyl- alanine | furan-2- carbonyl | 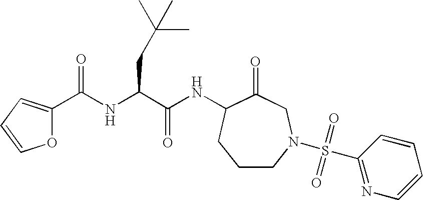 | (M + H) 491.2 |
| 11. | 2-pyridinyl | L-t-butyl- alanine | furan-3- carbonyl | 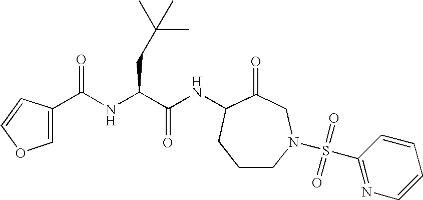 | (M + H) 491.2 |
| 12. | 2-pyridinyl | L-t-butyl- alanine | thieno- [3,2-β]- thiophene- 2-carbonyl | 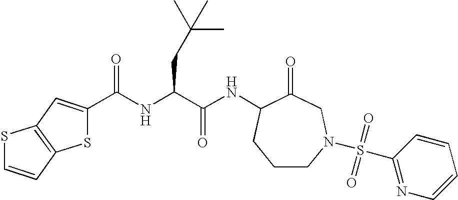 | (M + H) 563.2 |
| 13. | 2-pyridinyl | L-2- thiophenyl- alanine | 3-methyl- benzofuran- 2- carbonyl- | 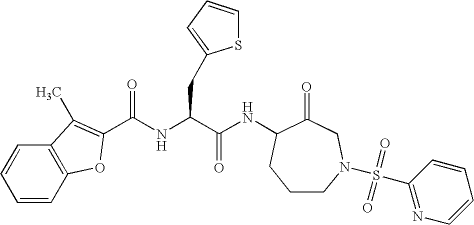 | (M + H) 581.4 |
| 14. | 2-pyridinyl | L-2- thiophenyl- alanine | benzofuran- 2- carbonyl- | 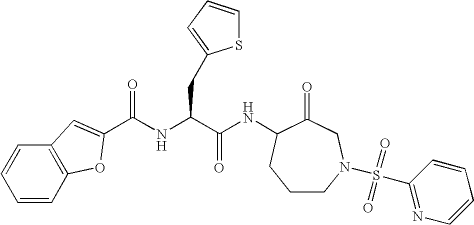 | (M + H) 567.4 |
| 15. | 2-pyridinyl | L-2- thiophenyl- alanine | 5-methoxy- benzofuran- 2- carbonyl- | 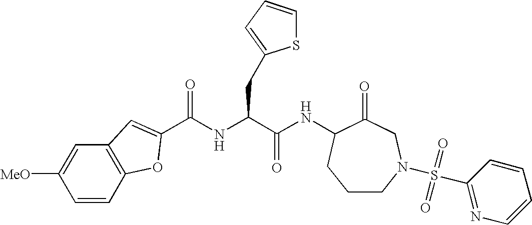 | (M + H) 597.4 |
| 16. | 2-pyridinyl | L-2- thiophenyl- alanine | benzothio- phene-2- carbonyl- | 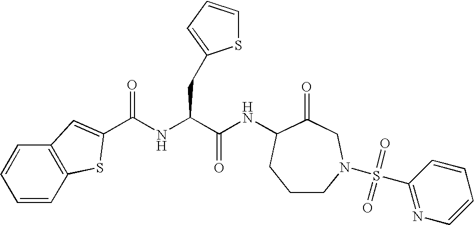 | (M + H) 583.2 |
| 17. | 2-pyridinyl | L-2- thiophenyl- alanine | quinoline- 2-carbonyl | 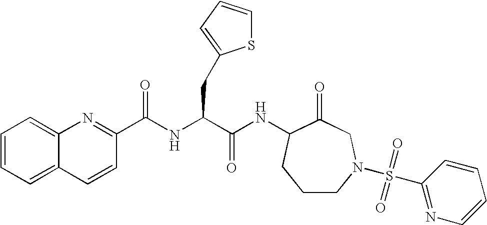 | (M + H) 578.4 |
| 18. | 2-pyridinyl | L-2- thiophenyl- alanine | quinoline- 3-carbonyl | 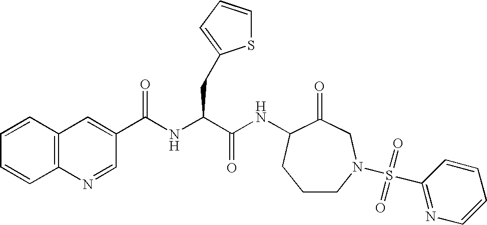 | (M + H) 578.4 |
| 19. | 2-pyridinyl | L-2- thiophenyl- alanine | thiophene- 2-carbonyl | 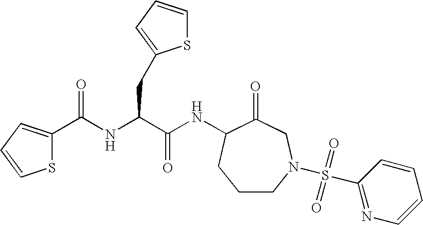 | (M + H) 533.2 |
| 20. | 2-pyridinyl | L-2- thiophenyl- alanine | thiophene- 3-carbonyl | 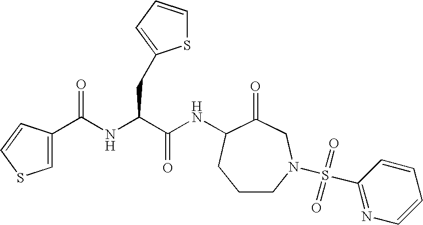 | (M + H) 533.2 |
| 21. | 2-pyridinyl | L-2- thiophenyl- alanine | 5- methylthio phene-2- carbonyl | 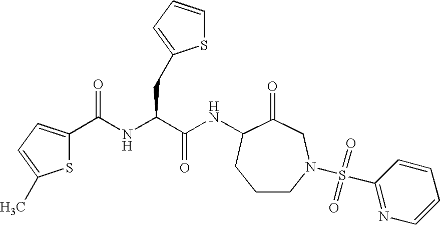 | (M + H) 547.2 |
| 22. | 2-pyridinyl | L-2- thiophenyl- alanine | furan-2- carbonyl | 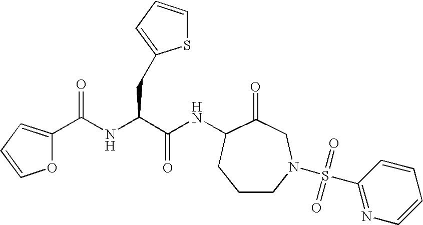 | (M + H) 517.2 |
| 23. | 2-pyridinyl | L-2- thiophenyl- alanine | furan-3- carbonyl | 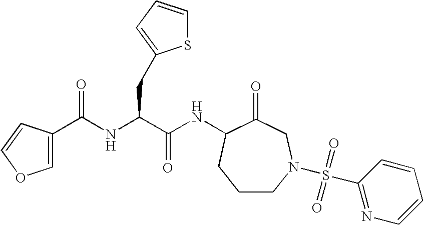 | (M + H) 517.2 |
| 24. | 2-pyridinyl | L-2- thiophenyl- alanine | thieno- [3,2-β]- thiophene- 2-carbonyl | 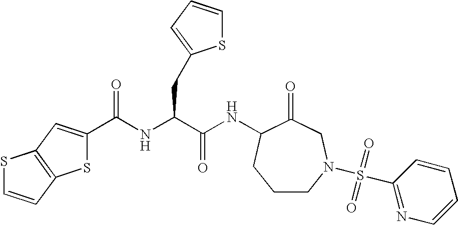 | (M + H) 589.2 |
| 25. | 2-pyridinyl | L- cyclohexyl- glycine | 3-methyl- benzofuran- 2- carbonyl- | 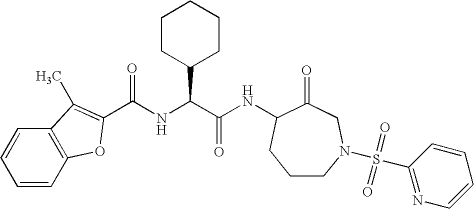 | (M + H) 567.2 |
| 26. | 2-pyridinyl | L- cyclohexyl- glycine | benzofuran- 2- carbonyl- | 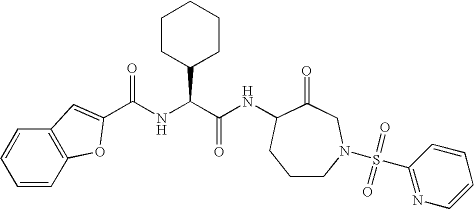 | (M + H) 533.4 |
| 27. | 2-pyridinyl | L- cyclohexyl- glycine | 5-methoxy- benzofuran- 2- carbonyl- | 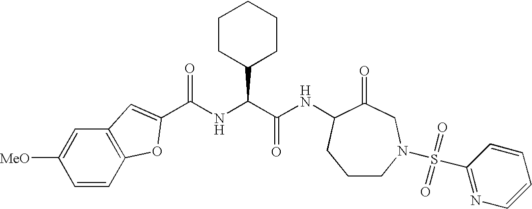 | (M + H) 583.4 |
| 28. | 2-pyridinyl | L- cyclohexyl- glycine | benzothio- phene-2- carbonyl- | 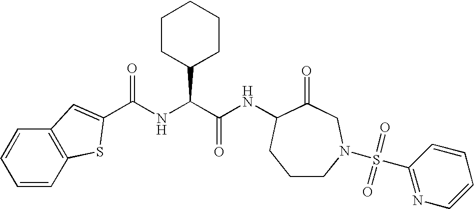 | (M + H) 569.4 |
| 29. | 2-pyridinyl | L- cyclohexyl- glycine | quinoline- 2-carbonyl | 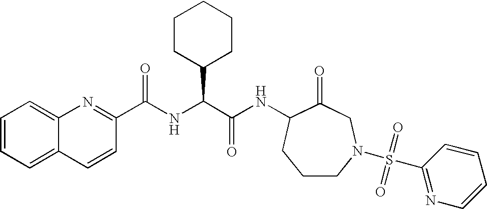 | (M + H) 564.2 |
| 30. | 2-pyridinyl | L- cyclohexyl- glycine | quinoline- 3-carbonyl | 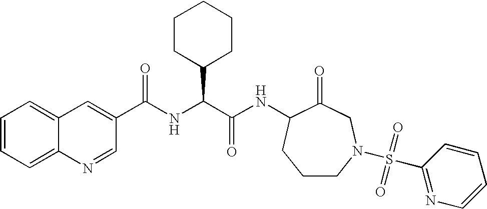 | (M + H) 564.2 |
| 31. | 2-pyridinyl | L- cyclohexyl- glycine | thiophene- 2-carbonyl | 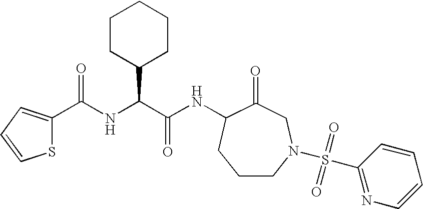 | (M + H) 519.2 |
| 32. | 2-pyridinyl | L- cyclohexyl- glycine | thiophene- 3-carbonyl | 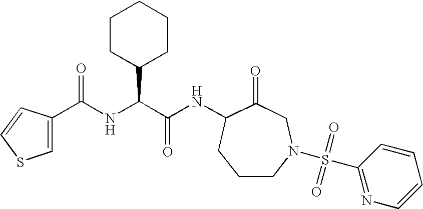 | (M + H) 519.2 |
| 33. | 2-pyridinyl | L- cyclohexyl- glycine | 5- methylthio phene-2- carbonyl | 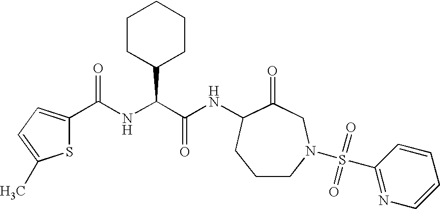 | (M + H) 533.2 |
| 34. | 2-pyridinyl | L- cyclohexyl- glycine | furan-2- carbonyl | 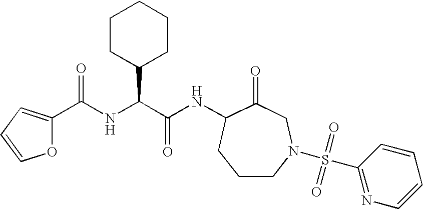 | (M + H) 563.2 |
| 35. | 2-pyridinyl | L- cyclohexyl- glycine | furan-3- carbonyl | 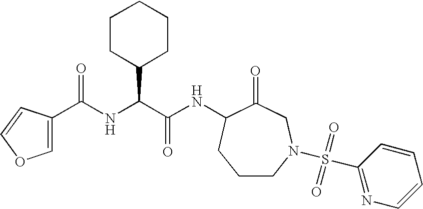 | (M + H) 503.0 |
| 36. | 2-pyridinyl | L- cyclohexyl- glycine | thieno- [3,2-β]- thiophene- 2-carbonyl | 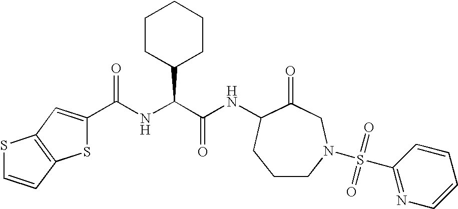 | (M + H) 575.4 |
| 37. | 2-pyridinyl | L-allo- isoleucine | 3-methyl- benzofuran- 2- carbonyl- | 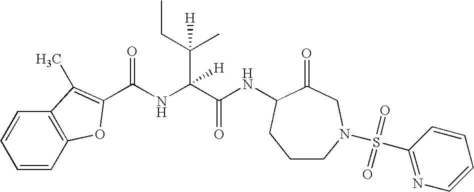 | (M + H) 541.4 |
| 38. | 2-pyridinyl | L-allo- isoleucine | benzofuran- 2- carbonyl- | 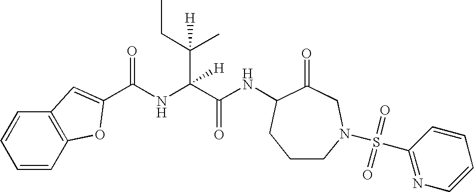 | (M + H) 527.2 |
| 39. | 2-pyridinyl | L-allo- isoleucine | 5-methoxy- benzofuran- 2- carbonyl- | 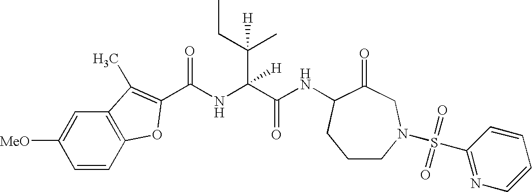 | (M + H) 557.2 |
| 40. | 2-pyridinyl | L-allo- isoleucine | benzothio- phene-2- carbonyl- | 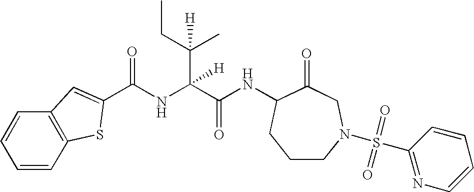 | (M + H) 543.2 |
| 41. | 2-pyridinyl | L-allo- isoleucine | quinoline- 2-carbonyl | 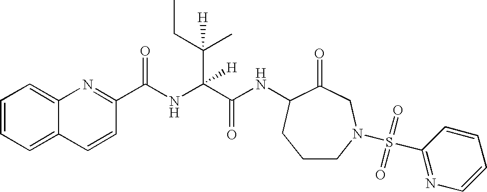 | (M + H) 538.2 |
| 42. | 2-pyridinyl | L-allo- isoleucine | quinoline- 3-carbonyl- | 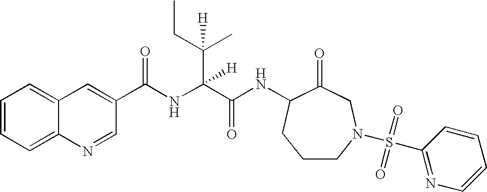 | (M + H) 538.2 |
| 43. | 2-pyridinyl | L-allo- isoleucine | thiophene- 2-carbonyl | 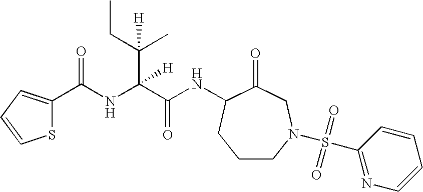 | (M + H) 493.2 |
| 44. | 2-pyridinyl | L-allo- isoleucine | thiophene- 3-carbonyl | 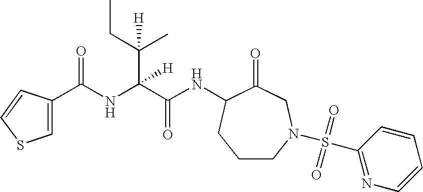 | (M + H) 493.2 |
| 45. | 2-pyridinyl | L-allo- isoleucine | 5- methylthio phene-2- carbonyl | 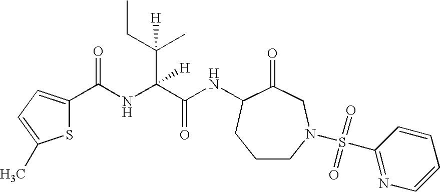 | (M + H) 507.2 |
| 46. | 2-pyridinyl | L-allo- isoleucine | furan-2- carbonyl | 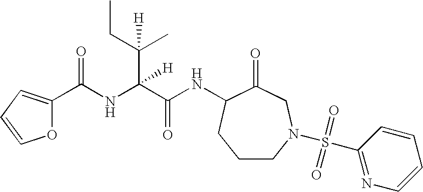 | (M + H) 477.2 |
| 47. | 2-pyridinyl | L-allo- isoleucine | furan-3- carbonyl | 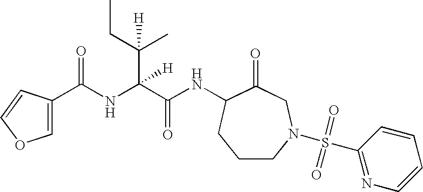 | (M + H) 477.2 |
| 48. | 2-pyridinyl | L-allo- isoleucine | thieno- [3,2-β]- thiophene- 2-carbonyl | 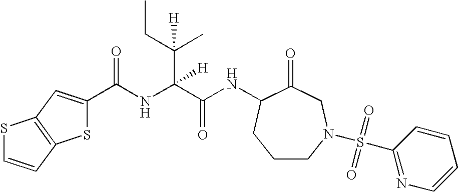 | (M + H) 549.2 |
| 49. | 2-pyridinyl | 1,2,3,4- Tetrahydro- isoquinoline- 3- carbonyl- | 3-methyl- benzofuran- 2- carbonyl- | 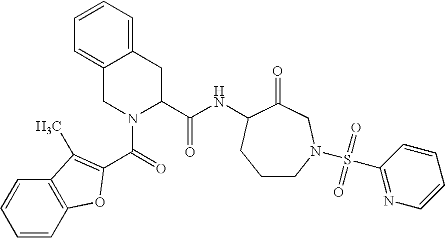 | (M + H) 587.2 |
| 50. | 2-pyridinyl | 1,2,3,4- Tetrahydro- isoquinoline- 3- carbonyl- | benzofuran- 2- carbonyl- | 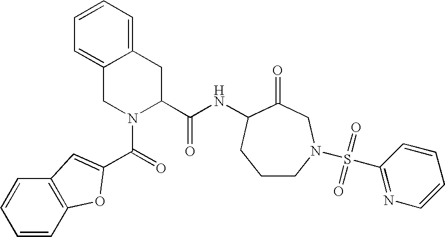 | (M + H) 573.2 |
| 51. | 2-pyridinyl | 1,2,3,4- Tetrahydro- isoquinoline- 3- carbonyl- | 5-methoxy- benzofuran- 2- carbonyl- | 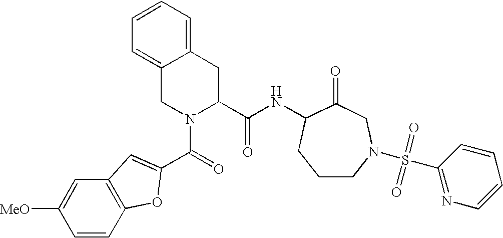 | (M + H) 603.4 |
| 52. | 2-pyridinyl | 1,2,3,4- Tetrahydro- isoquinoline- 3- carbonyl- | benzothio- phene-2- carbonyl- | 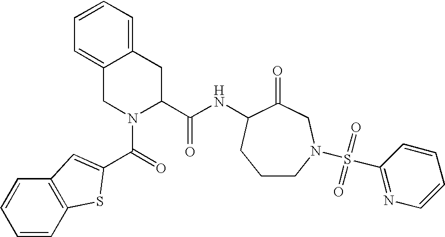 | (M + H) 589.2 |
| 53. | 2-pyridinyl | 1,2,3,4- Tetrahydro- isoquinoline- 3- carbonyl- | quinoline- 2-carbonyl | 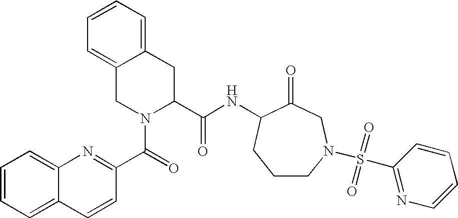 | (M + H) 584.4 |
| 54. | 2-pyridinyl | 1,2,3,4- Tetrahydro- isoquinoline- 3- carbonyl- | quinoline- 3-carbonyl | 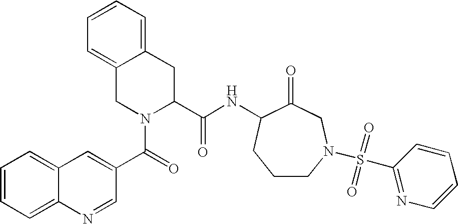 | (M + H) 584.2 |
| 55. | 2-pyridinyl | 1,2,3,4- Tetrahydro- isoquinoline- 3- carbonyl- | thiophene- 2-carbonyl | 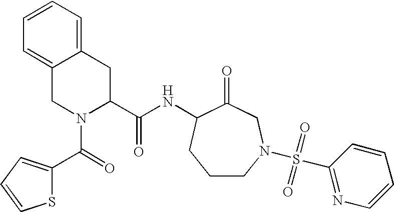 | (M + H) 539.2 |
| 56. | 2-pyridinyl | 1,2,3,4- Tetrahydro- isoquinoline- 3- carbonyl- | thiophene- 3-carbonyl | 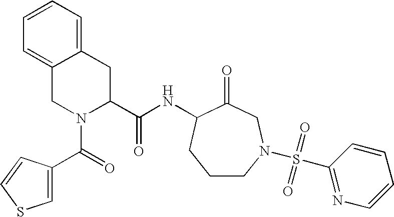 | (M + H) 539.2 |
| 57. | 2-pyridinyl | 1,2,3,4- Tetrahydro-= isoquinoline- 3- carbonyl- | 5- methylthio phene-2- carbonyl | 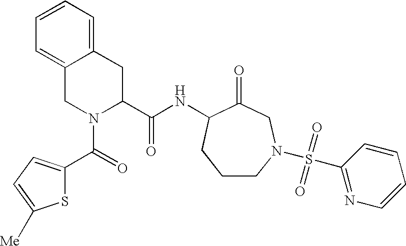 | (M + H) 553.4 |
| 58. | 2-pyridinyl | 1,2,3,4- Tetrahydro- isoquinoline- 3- carbonyl- | furan-2- carbonyl | 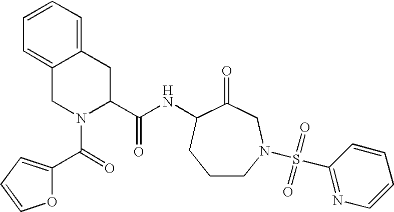 | (M + H) 523.4 |
| 59. | 2-pyridinyl | 1,2,3,4- Tetrahydro- isoquinoline- 3- carbonyl- | furan-3- carbonyl | 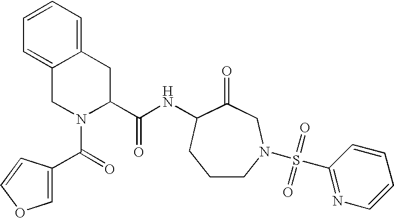 | (M + H) 523.2 |
| 60. | 2-pyridinyl | 1,2,3,4- Tetrahydro- isoquinoline- 3- carbonyl- | thieno- [3,2-β]- thiophene- 2-carbonyl | 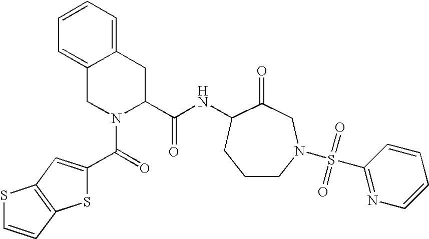 | (M + H) 595.4 |
| 61. | 2-pyridinyl | L-proline- | 3-methyl- benzofuran- 2- carbonyl- | 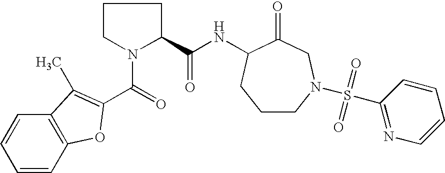 | (M + H) 525.2 |
| 62. | 2-pyridinyl | L-proline- | benzofuran- 2- carbonyl- | 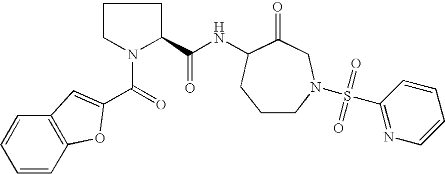 | (M + H) 511.2 |
| 63. | 2-pyridinyl | L-proline- | 5-methoxy- benzofuran- 2- carbonyl- | 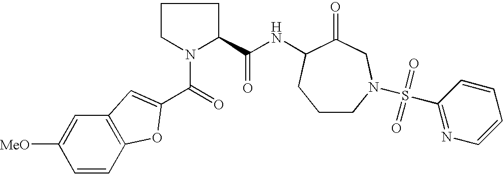 | (M + H) 541.2 |
| 64. | 2-pyridinyl | L-proline- | benzothiop hene-2- carbonyl- | 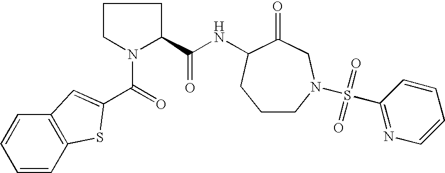 | (M + H) 527.2 |
| 65. | 2-pyridinyl | L-proline- | quinoline- 2-carbonyl- | 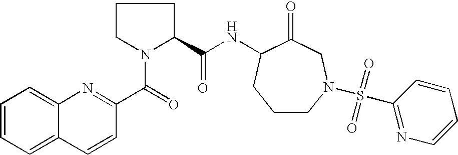 | (M + H) 522.2 |
| 66. | 2-pyridinyl | L-proline- | quinoline- 3-carbonyl- | 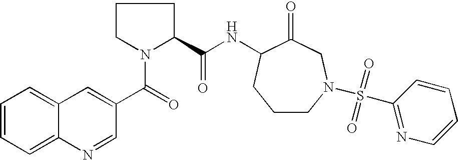 | (M + H) 522.2 |
| 67. | 2-pyridinyl | L-proline- | thiophene- 2-carbonyl- | 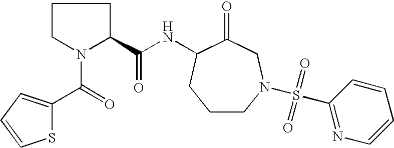 | (M + H) 477.0 |
| 68. | 2-pyridinyl | L-proline- | thiophene- 3-carbonyl- | 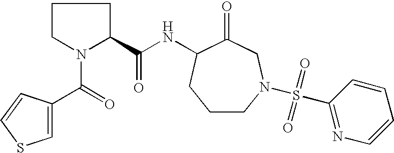 | (M + H) 477.0 |
| 69. | 2-pyridinyl | L-proline- | 5- methylthio phene-2- carbonyl | 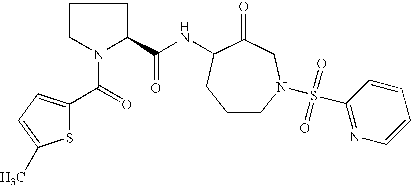 | (M + H) 491.2 |
| 70. | 2-pyridinyl | L-proline- | furan-2- carbonyl | 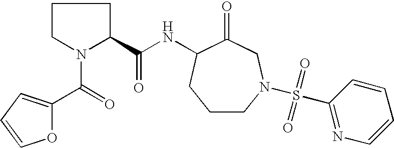 | (M + H) 461.0 |
| 71. | 2-pyridinyl | L-proline- | furan-3-0 carbonyl | 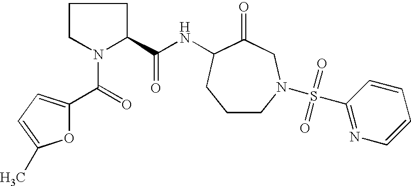 | (M + H) 461.0 |
| 72. | 2-pyridinyl | L-proline- | thieno- [3,2-β]- thiophene- 2-carbonyl- | 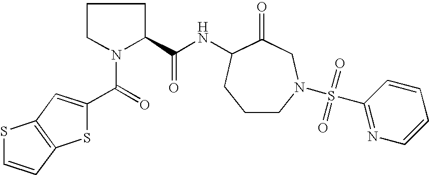 | (M + H) 533.2 |
| 73. | 2-pyridinyl | (S)-2- Amino-4- methanesu lfonyl- butanoyl- | 3-methyl- benzofuran- 2- carbonyl | 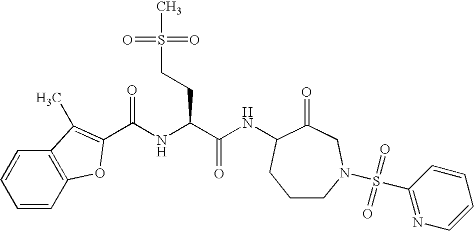 | (M + H) 591.2 |
| 74. | 2-pyridinyl | (S)-2- Amino-4- methanesu lfonyl- butanoyl- | benzofuran- 2- carbonyl- | 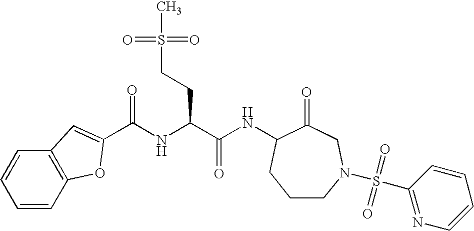 | (M + H) 577.2 |
| 75. | 2-pyridinyl | (S)-2- Amino-4- methanesu lfonyl- butanoyl | 5-methoxy- benzofuran- 2- carbonyl- | 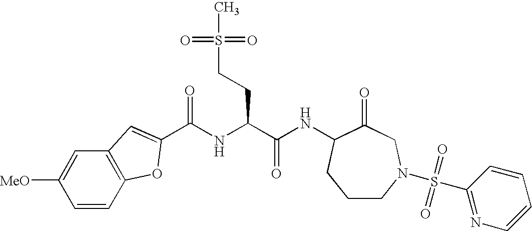 | (M + H) 607.4 |
| 76. | 2-pyridinyl | (S)-2- Amino-4- methanesu lfonyl- butanoyl- | benzothiop hene-2- carbonyl- | 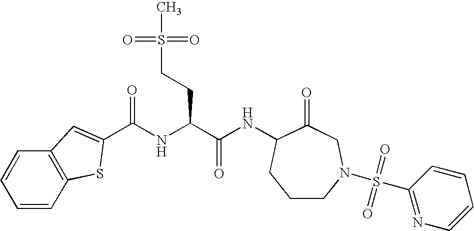 | (M + H) 593.4 |
| 77. | 2-pyridinyl | (S)-2- Amino-4- methanesu lfonyl- butanoyl- | quinoline- 2-carbonyl- | 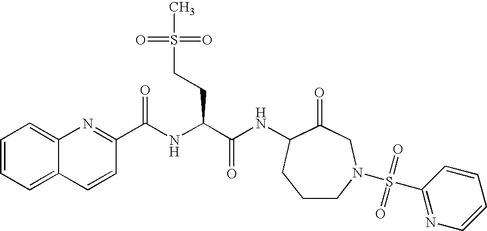 | (M + H) 588.4 |
| 78. | 2-pyridinyl | (S)-2- Amino-4- methanesu- lfonyl- butanoyl- | quinoline- 3-carbonyl- | 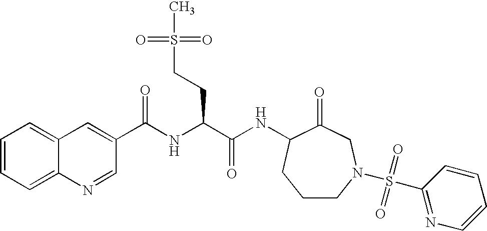 | (M + H) 588.4 |
| 79. | 2-pyridinyl | (S)-2- Amino-4- methanesu lfonyl- butanoyl- | quinoline- 3-carbonyl- | 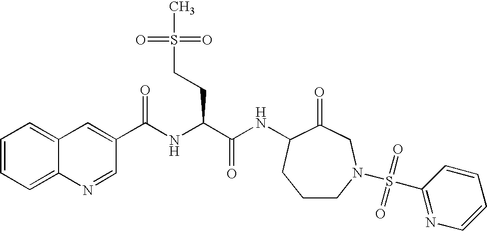 | (M + H) 588.4 |
| 80. | 2-pyridinyl | (S)-2- Amino-4- methanesu lfonyl- butanoyl- | thiophene- 3-carbonyl- | 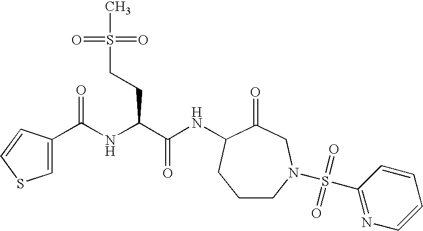 | (M + H) 543.2 |
| 81. | 2-pyridinyl | (S)-2- Amino-4- methanesu lfonyl- butanoyl- | 5- methylthio phene-2- carbonyl- | 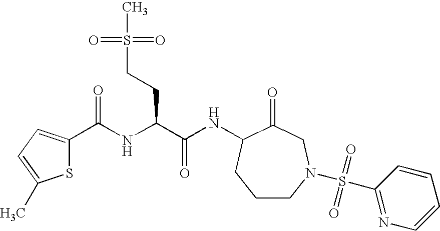 | (M + H) 557.2 |
| 82. | 2-pyridinyl | (S)-2- Amino-4- methanesu lfonyl- butanoyl- | furan-2- carbonyl- | 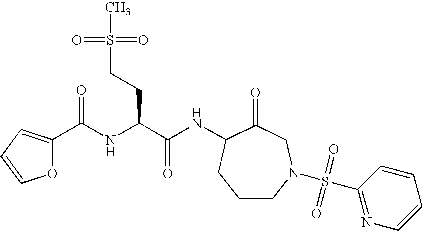 | (M + H) 527.2 |
| 83. | 2-pyridinyl | (S)-2- Amino-4- methanesu lfonyl- butanoyl- | furan-3- carbonyl- | 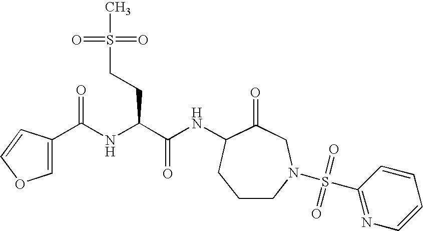 | (M + H) 527.2 |
| 84. | 2-pyridinyl | (S)-2- Amino-4- methanesu lfonyl- butanoyl- | thieno- [3,2-β]- thiophene- 2-carbonyl | 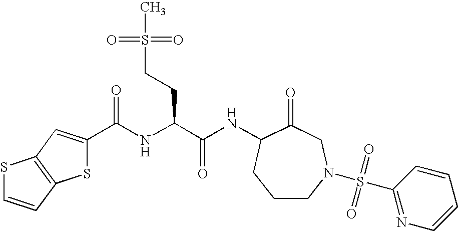 | (M + H) 599.2 |
| 85. | 2-pyridinyl | (S)- Piperidine- 2- carbonyl- | benzofuran- 2- carbonyl- | 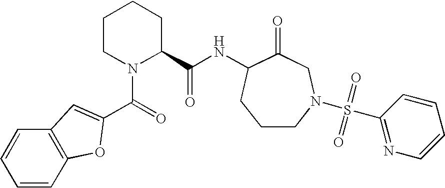 | (M + H) 525.2 |
| 86. | 2-pyridinyl | (S)- Piperidine- 2- carbonyl- | benzothiop hene-2- carbonyl- | 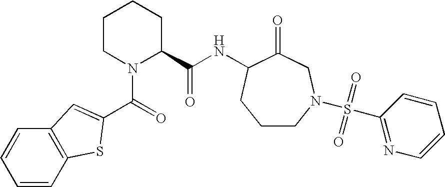 | (M + H) 541.2 |
| 87. | 2-pyridinyl | (S)- Piperidine- 2- carbonyl- | 3-methyl- benzofuran- 2- carbonyl- | 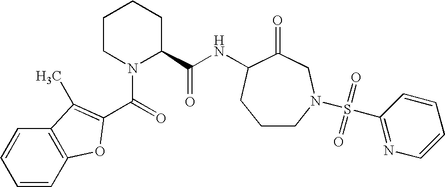 | (M + H) 539.2 |
| 88. | 2-pyridinyl | (S)- Piperidine- 2- carbonyl- | 5-methoxy- benzofuran- 2- carbonyl- | 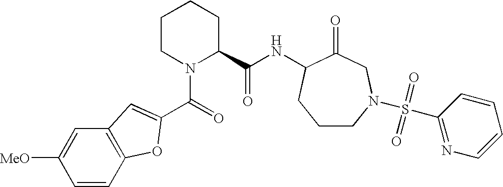 | (M + H) 555.2 |
| 89. | 2-pyridinyl | (S) Piperidine- 2- carbonyl- | quinoline- 2-carbonyl | 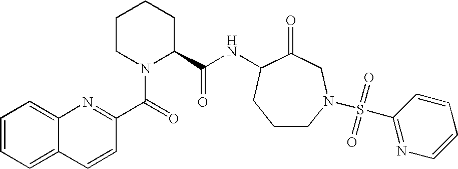 | (M + H) 536.2 |
| 90. | 2-pyridinyl | (S)- Piperidine- 2- carbonyl- | quinoline- 3-carbonyl- | 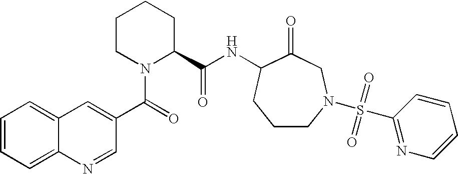 | (M + H) 536.2 |

The above specification and Examples fully disclose how to make and use the compounds of the present invention. However, the present invention is not limited to the particular embodiments described hereinabove, but includes all modifications thereof within the scope of the following claims. The various references to journals, patents and other publications which are cited herein comprise the state of the art and are incorporated herein by reference as though fully set forth.

We claim:

1. A compound of Formula I: 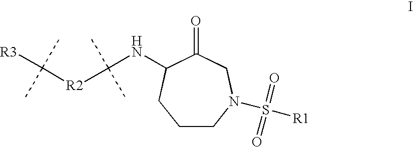
wherein:
R¹ is 2-pyridinyl;
R² is selected from the group consisting of: L-t-butyl-alaninyl, L-2-thiophenyl-alaninyl, L-cyclohexyl-glycinyl, L-allo-isoleucinyl, 1,2,3,4-tetrahydro-isoquinoline-3-carbonyl, L-prolinyl, (S)-2-amino-4-methanesulfonyl-butanoyl, and (S)-piperidine-2-carbonyl;
R³ is selected from the group consisting of: 3-methyl-benzofuran-2-carbonyl, benzofuran-2-carbonyl, 5-methoxy-benzofuran-2-carbonyl, benzothiophene-2-carbonyl, quinoline-2-carbonyl, quinoline-3-carbonyl, thiophene-2-carbonyl, thiophene-3-carbonyl, 5-methylthiophene-2-carbonyl, furan-2-carbonyl, furan-3-carbonyl, and thieno-[3,2-β]-thiophene-2-carbonyl;
and pharmaceutically acceptable salts, hydrates or solvates thereof.

2. A compound according to claim 1 selected from the group consisting of:
quinoline-2-carboxylic acid {(S)-3,3-dimethyl-1-[(S)-3-oxo-1-(pyridine-2-sulfonyl)-azepan-4-ylcarbamoyl]-butyl}-amide;
benzofuran-2-carboxylic acid {(S)-3,3-dimethyl-1-[(S)-3-oxo-1-(pyridine-2-sulfonyl)-azepan-4-ylcarbamoyl]-butyl}-amide;
5-methoxy-benzofuran-2-carboxylic acid {(S)-3,3-dimethyl-1-[(S)-3-oxo-1-(pyridine-2-sulfonyl)-azepan-4-ylcarbamoyl]-butyl}-amide;
benzo[b]thiophene-2-carboxylic acid {(S)-3,3-dimethyl-1-[(R)-3-oxo-1-(pyridine-2-sulfonyl)-azepan-4-ylcarbamoyl]-butyl}-amide;
thiophene-2-carboxylic acid {(S)-3,3-dimethyl-1-[(S)-3-oxo-1-(pyridine-2-sulfonyl)-azepan-4-ylcarbamoyl]-butyl)-amide;
5-methyl-thiophene-2-carboxylic acid {(S)-3,3-dimethyl-1-[(S)-3-oxo-1-(pyridine-2-sulfonyl)-azepan-4-ylcarbamoyl]-butyl}-amide;
furan-2-carboxylic acid {(S)-3,3-dimethyl-1-[(S)-3-oxo-1-(pyridine-2-sulfonyl)-azepan-4-ylcarbamoyl]-butyl}-amide;
furan-3-carboxylic acid {(S)-3,3-dimethyl-1-[(S)-3-oxo-1-(pyridine-2-sulfonyl)-azepan-4-ylcarbamoyl]-butyl}-amide;
thieno[3,2-b]thiophene-2-carboxylic acid {(S)-3,3-dimethyl-1-[(S)-3-oxo-1-(pyridine-2-sulfonyl)-azepan-4-ylcarbamoyl]-butyl}-amide;
thieno[3,2-b]thiophene-2-carboxylic acid {(S)-1-[(S)-3-oxo-1-(pyridine-2-sulfonyl)-azepan-4-ylcarbamoyl]-2-thiophen-2-yl-ethyl}-amide;
thieno[3,2-b]thiophene-2-carboxylic acid {(S)-1-cyclohexyl-1-[(S)-3-oxo-1-(pyridine-2-sulfonyl)-azepan-4-ylcarbamoyl]-methyl}-amide; and
thieno[3,2-b]thiophene-2-carboxylic acid {(1S,2R)-2-methyl-1-[(S)-3-oxo-1-(pyridine-2-sulfonyl)-azepan-4-ylcarbamoyl]-butyl }-amide.

3. A pharmaceutical composition comprising a compound according to any one of claims 1 or 2 and a pharmaceutically acceptable carrier, diluent or excipient.

4. A method of inhibiting a protease, comprising administering to a patient in need thereof an effective amount of a compound according to any one of claims 1 or 2.

5. A method according to claim 4 wherein said protease is selected from the group consisting of a cysteine protease and a serine protease.

6. A method according to claim 5 wherein said protease is a cysteine protease.

7. A method according to claim 6 wherein said cysteine protease is cathepsin K.

8. A method of treating a disease characterized by bone loss comprising inhibiting said bone loss by administering to a patient in need thereof an effective amount of a compound according to any one of claims 1 or 2.

9. A method according to claim 8 wherein said disease is osteoporosis.

10. A method according to claim 8 wherein said disease is periodontitis.

11. A method according to claim 8 wherein said disease is gingivitis.

12. A method of treating a disease characterized by excessive cartilage or matrix degradation comprising inhibiting said excessive cartilage or matrix degradation by administering to a patient in need thereof an effective amount of a compound according to claims 1 or 2.

13. A method according to claim 12 wherein said disease is osteoarthritis.

14. A method according to claim 12 wherein said disease is rheumatoid arthritis.

15. Use of a compound according to any one of claims 1 or 2 in the manufacture of a medicament for use in inhibiting a protease selected from the group consisting of a cysteine protease and a serine protease.

16. A use according to claim 15 wherein said protease is a cysteine protease.

17. A use according to claim 16 wherein said cysteine protease is cathepsin K.

18. Use of a compound according to any one of claims 1 or 2 in the manufacture of a medicament for use in treating a disease characterized by bone loss.

19. A use according to claim 18 wherein said disease is osteoporosis.

20. A use according to claim 18 wherein said disease is periodontitis.

21. A use according to claim 18 wherein said disease is gingivitis.

22. Use of a compound according to any one of claims 1 or 2 in the manufacture of a medicament for use in treating a disease characterized by excessive cartilage or matrix degradation.

23. A use according to claim 22 wherein said disease is osteoarthritis.

24. A use according to claim 22 wherein said disease is rheumatoid arthritis.
